(19) United States
(12) Patent Application Publication
(10) Pub. No.: US 2012/0108964 A1
(43) Pub. Date: May 3, 2012

(54) PERFORMING GAIN CONTROL PROCESS BASED ON APPLICATION IN ULTRASOUND SYSTEM

(75) Inventors: Jae Keun LEE, Seoul (KR); Hyeong Do Lee, Chuncheon-si (KR)

(73) Assignee: SAMSUNG MEDISON CO., LTD.

(21) Appl. No.: 13/286,731

(22) Filed: Nov. 1, 2011

(30) Foreign Application Priority Data
Nov. 3, 2010 (KR) 10-2010-0108521

(51) Int. Cl. A61B 8/00

(52) U.S. Cl. 600/437

(57) ABSTRACT

Embodiments for performing a gain control process based on information of at least one gain control curve corresponding to at least one application are disclosed herein. In one embodiment, by way of non-limiting example, an ultrasound system comprises: an ultrasound data acquisition unit configured to acquire ultrasound data corresponding to a living body; a storage unit for storing information of at least one gain control curve corresponding to at least one application; and a processing unit configured to extract information a gain control curve corresponding to input information for selecting an application from the storage unit, form a gain control curve based on the extracted information, and perform a gain control process upon the ultrasound data based on the gain control curve.

### CROSS-REFERENCE TO RELATED APPLICATION

The present application claims priority from Korean Patent Application No. 10-2010-0108521 filed on Nov. 3, 2010, the entire subject matter of which is incorporated herein by reference.

### TECHNICAL FIELD

The present disclosure generally relates to ultrasound systems, and more particularly to performing a gain control process based on an application in an ultrasound system.

### BACKGROUND

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two-dimensional or three-dimensional ultrasound images of internal features of a target object (e.g., human organs).

The ultrasound system may transmit ultrasound signals to a living body, which includes a target object (e.g., a heart, a fetus, etc.), and receive ultrasound signals (i.e., ultrasound echo signals) from the living body to thereby form ultrasound data. The ultrasound system may further form an ultrasound image based on the ultrasound data.

The ultrasound system may perform a gain control process upon the ultrasound data to improve the resolution of an ultrasound image. The ultrasound system may perform the gain control process upon the ultrasound data by applying a transfer function of a linear or curve type to the ultrasound data without reference to an application. However, there is a problem since the ultrasound image may not be represented naturally by the gain control process.

### SUMMARY

Embodiments for performing a gain control process based on information of at least one gain control curve corresponding to at least one application in an ultrasound system are disclosed herein.

In one embodiment, by way of non-limiting example, an ultrasound system comprises: an ultrasound data acquisition unit configured to acquire ultrasound data corresponding to a living body; a storage unit for storing information of at least one gain control curve corresponding to at least one application; and a processing unit configured to extract information a gain control curve corresponding to input information for selecting an application from the storage unit, form a gain control curve based on the extracted information, and perform a gain control process upon the ultrasound data based on the gain control curve.

In another embodiment, there is a method of performing a gain control process, comprising: a) acquiring ultrasound data corresponding to a living body; b) extracting information a gain control curve corresponding to input information for selecting an application from a storage unit for storing information of at least one gain control curve corresponding to at least one application; c) forming a gain control curve based on the extracted information; and d) performing a gain control process upon the ultrasound data based on the gain control curve.

This summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a block diagram showing an illustrative embodiment of an ultrasound data acquisition unit.

FIG. 3 is a schematic diagram showing an example of a gain control curve.

FIG. 4 is a flow chart showing a process of performing a gain control process.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

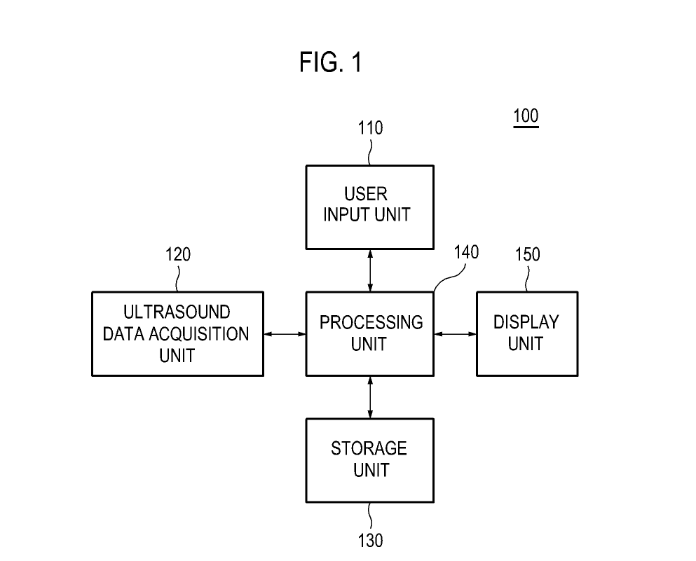

Referring to FIG. 1, an ultrasound system 100 in accordance with an illustrative embodiment is shown. As depicted therein, the ultrasound system 100 may include a user input unit 110.

The user input unit 110 may be configured to receive input information from a user. In one embodiment, the input information may include application selection information for selecting an application. However, it should be noted herein that the input information may not be limited thereto. The application may represent a diagnostic part (i.e., target object) of a living body. The target object may include a heart, a fetus and the like. The user input unit 110 may include a control panel, a trackball, a mouse, a keyboard and the like.

The ultrasound system 100 may further include an ultrasound data acquisition unit 120. The ultrasound data acquisition unit 120 may be configured to transmit ultrasound signals to the living body and receive ultrasound signals (i.e., ultrasound echo signals) from the living body to acquire ultrasound data.

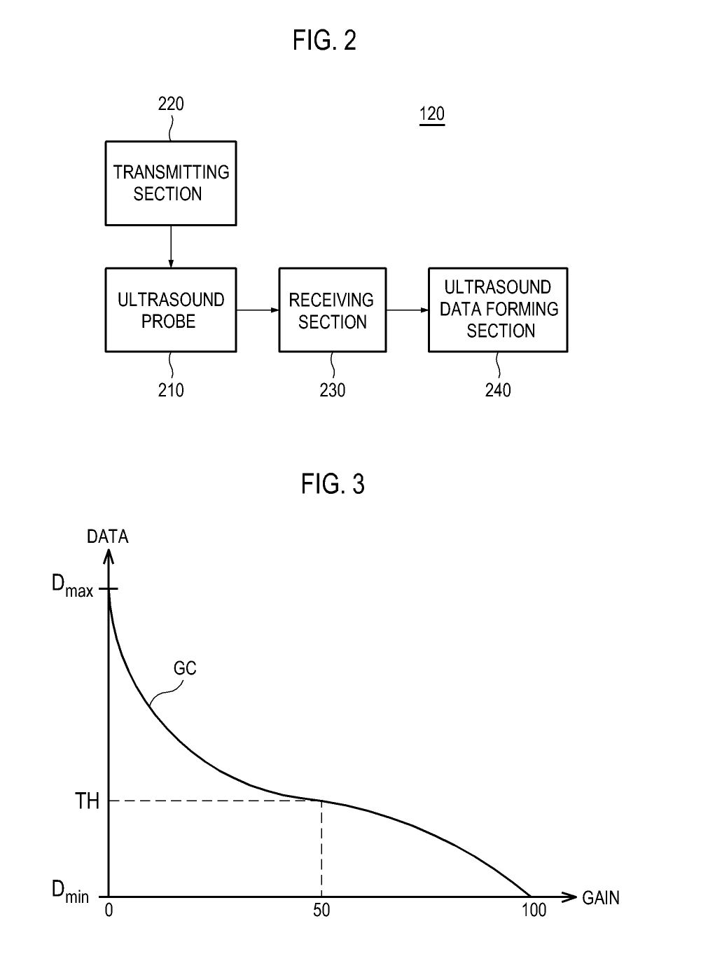

FIG. 2 is a block diagram showing an illustrative embodiment of the ultrasound data acquisition unit. Referring to FIG. 2, the ultrasound data acquisition unit 120 may include an ultrasound probe 210.

The ultrasound probe 210 may include a plurality of elements (not shown) for reciprocally converting between ultrasound signals and electrical signals. The ultrasound probe 210 may be configured to transmit ultrasound signals to the living body. The ultrasound probe 210 may be further configured to receive ultrasound echo signals from the living body to thereby output received signals. The received signals may be analog signals. The ultrasound probe 210 may include a convex probe, a linear probe, a three-dimensional mechanical probe and the like.

The ultrasound data acquisition unit 120 may further include a transmitting section 220. The transmitting section 220 may be configured to control the transmission of the ultrasound signals. The transmitting section 220 may be further configured to generate electrical signals (“transmitting signals”) for obtaining an ultrasound image in consideration of the elements and focal points. Thus, the ultrasound probe 210 may be configured to convert the transmitting signals into the ultrasound signals, transmit the ultrasound signals to the living body and receive the ultrasound echo signals from the living body to thereby output the received signals. The ultrasound image may include a brightness mode image, a three-dimensional mode image and the like. The transmitting section 220 may include a transmitting signal forming section (not shown), a transmitting delay time information memory (not shown), a transmitting beam former (not shown) and the like.

The ultrasound data acquisition unit 120 may further include a receiving section 230. The receiving section 230 may be configured to convert the received signals provided from the ultrasound probe 210 into digital signals. The receiving section 230 may be further configured to apply delays to the digital signals in consideration of the elements and the focal points to thereby output digital receive-focused signals. The receiving section 230 may include an analog-to-digital converter (not shown), a receiving delay time information memory (not shown), a receiving beam former (not shown) and the like.

The ultrasound data acquisition unit 120 may further include an ultrasound data forming section 240. The ultrasound data forming section 240 may be configured to form ultrasound data corresponding to the ultrasound image based on the digital receive-focused signals provided from the receiving section 230. The ultrasound data may include radio frequency data. However, it should be noted herein that the ultrasound data may not be limited thereto. The ultrasound data forming section 240 may be further configured to perform signal processing upon the digital receive-focused signals.

Referring back to FIG. 1, the ultrasound system 100 may further include a storage unit 130. The storage unit 130 may store information of at least one gain control curve corresponding to at least one application. In one embodiment, the information of the at least one gain control curve may include a threshold value TH for discriminating a noise, a maximum value Dₘₐₓ of input data (i.e., ultrasound data), a minimum value Dₘᵢₙ of the input data and a curve characteristic value for at least one application, as shown in FIG. 3. The threshold value, the maximum value and the minimum value may be set variously by a user. The curve characteristic value may be a value, which represents a sigmoid degree of the gain control curve GC based on the threshold value TH. When the curve characteristic value is 1, the gain control curve may become a straight line. When the curve characteristic value is not 1, the sigmoid degree of the gain control curve may be set by the curve characteristic value. The methods of setting the sigmoid degree based on the curve characteristic value are well known in the art. Thus, they have not been described in detail so as not to unnecessarily obscure the present invention.

The ultrasound system 100 may further include a processing unit 140 in communication with the user input unit 110, the ultrasound data acquisition unit 120 and the storage unit 130. The processing unit 140 may include a central processing unit, a microprocessor, a graphic processing unit and the like.

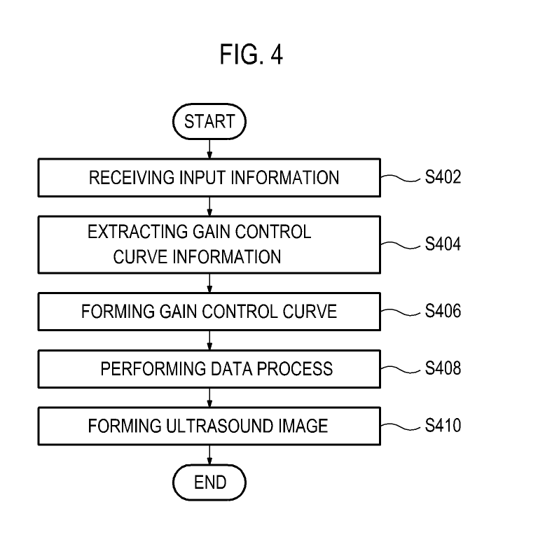

FIG. 4 is a flow chart showing a process of performing a gain control process. The user input unit 110 may be configured to receive the input information, at step S402 in FIG. 4. The processing unit 140 may be configured to retrieve the storage unit 130 to extract information of a gain control curve corresponding to the input information provided from the user input unit 110, at step S404 in FIG. 4.

The processing unit 140 may be configured to form the gain control curve as shown in FIG. 3 based on the extracted gain control curve information, at step S406 in FIG. 4. The methods of forming the gain control curve are well known in the art. Thus, they have not been described in detail so as not to unnecessarily obscure the present invention.

The processing unit 140 may be configured to perform data processing (i.e., gain control process) upon the ultrasound data provided from the ultrasound data acquisition unit 120 based on the gain control curve, at step S408 in FIG. 4. The processing unit 140 may be configured to form the ultrasound image based on the data-processed (i.e., gain control processed) ultrasound data, at step S410 in FIG. 4.

Referring back to FIG. 1, the ultrasound system 100 may further include a display unit 150. The display unit 150 may be configured to display the ultrasound image formed by the processing unit 140.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
an ultrasound data acquisition unit configured to acquire ultrasound data corresponding to a living body;
a storage unit for storing information of at least one gain control curve corresponding to at least one application; and
a processing unit configured to extract information a gain control curve corresponding to input information for selecting an application from the storage unit, form a gain control curve based on the extracted information, and perform a gain control process upon the ultrasound data based on the gain control curve.

2. The ultrasound system of claim 1, wherein the gain control curve information includes a threshold value for discriminating a noise, a maximum value of the ultrasound data, a minimum value of the ultrasound data and a curve characteristic value for setting a sigmoid degree of the gain control curve based on the threshold value.

3. A method of performing a gain control process, comprising:
a) acquiring ultrasound data corresponding to a living body;
b) extracting information a gain control curve corresponding to input information for selecting an application from a storage unit for storing information of at least one gain control curve corresponding to at least one application;
c) forming a gain control curve based on the extracted information; and
d) performing a gain control process upon the ultrasound data based on the gain control curve.

4. The method of claim 3, wherein the gain control curve information includes a threshold value for discriminating a noise, a maximum value of the ultrasound data, a minimum value of the ultrasound data and a curve characteristic value for setting a sigmoid degree of the gain control curve based on the threshold value.
